SEQUENCE LISTING (7 sequences)

SEQ ID NO: 1 - DNA, 46 bp, Artificial Sequence
  synthetic dsRNA encoding sequence (luc1)
gaaacgatatgggctgaatacttaagtattcagcccatatcgtttc

SEQ ID NO: 2 - DNA, 46 bp, Artificial Sequence
  synthetic dsRNA encoding sequence
gatggtgcttcaagctagtacttaagtactagcttgaagcaccatc

SEQ ID NO: 3 - DNA, 48 bp, Artificial Sequence
  synthetic dsRNA encoding sequence
gatggtgcttcaagctagtacggatccgtactagcttgaagcaccatc

SEQ ID NO: 4 - DNA, 48 bp, Artificial Sequence
  synthetic dsRNA encoding sequence
gaaacgatatgggctgaatacggatccgtattcagcccatatcgtttc

SEQ ID NO: 5 - DNA, 46 bp, Artificial Sequence
  synthetic dsRNA encoding sequence
cctaataattatcaaaatgtggatccacattttgataattattagg

SEQ ID NO: 6 - DNA, 44 bp, Artificial Sequence
  synthetic dsRNA encoding sequence
cctaataattatcaaaatgtaattacattttgataattattagg

SEQ ID NO: 7 - DNA, 34383 bp, Artificial Sequence
  synthetic recombinant chimeric E1/E3 deleted adenovirus type 5 vector pAd-DX1
taaggatcccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgctagagatctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgatgggtaccgccaccatgaaggctatacttgtcgtactgctctacacgttcgctactgctaacgccgacaccctctgcattggataccatgccaataactctaccgatactgtggacacggtgctggaaaaaaacgtgacagtaacacactcagtgaaccttctcgaagacaagcacaatggcaaactgtgcaagctgcggggcgttgctcctctgcacctgggcaaatgtaacatagccggctggattcttggcaatccagagtgcgaatctttgagcaccgcaagcagctggagttacattgtggaaaccccgtcctccgataacggaacttgctacccaggagactttatcgactacgaggaactgagagagcagctcagctccgtgtcttccttcgaacgctttgagatcttcccaaaaacatcctcctggccaaaccacgatagtaacaagggggtaacagccgcgtgcccacatgcaggggctaaatcattttataagaacctcatctggctcgtgaagaagggtaacagctacccaaaactttctaagtcttatataaacgataagggcaaggaggttctggtcctgtggggtatacaccatccctctaccagtgcagaccagcaatcattgtatcagaacgctgacacatacgttttcgtaggctctagcagatattccaagaaattcaagccagagatcgccatcaggcctaaggtccgcgaccaggagggccgaatgaactattactggacactcgtcgaacctggggataagattacctttgaggccactggtaatctggttgttcctagatacgcattcgctatggagcgcaatgccggtagtggcatcataatctctgacacccccgttcacgactgtaataccacttgccagacccctaaaggagcaatcaatacctcattgcccttccagaatatccatcccatcacaataggaaagtgccctaaatacgtgaaaagcaccaagctgcggttggccactggtctgcggaatataccgagcatccagtctcgaggactcttcggggcaatcgctgggtttatcgagggcggctggaccgggatggtcgacggttggtacggataccaccatcagaacgagcaagggtccggctatgccgcagacctcaagtctacccagaacgccatcgatgagattacgaataaagtaaattccgtgatcgagaagatgaatacacagtttacagccgtggggaaggagttcaaccatctggaaaagcggattgaaaacctgaataaaaaagtggatgatggatttttggatatttggacttacaacgccgagctgctcgttctgctggagaatgaaagaaccctggattaccacgatagtaacgtgaagaacctttacgagaaagttaggagccaactgaagaataacgctaaagaaatcggcaacggttgctttgaattctaccacaagtgcgataacacatgcatggagtctgttaaaaatgggacttacgactatcctaaatactccgaggaagccaagttgaatagagaggagatcgatggagtcaagctcgagagcacgcgcatttaccagatactggccatttattccacagttgcgtcatccctggtgctcgttgtgagcttgggggccatatccttctggatgtgctctaacgggagcctccagtgtcgaatctgcatcggtaccggcggccgccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaaagcttagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctgggaaacgatatgggctgaatacggatccgtattcagcccatatcgtttctctagaaataaaatatctttattttcattacatctgtgtgttggttttttgtgtggcggccgctcgagcctaagcttctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagcttgtcgactcgaagaatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatcctctagttataactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatnnnnnttaat

(19) United States
(12) Patent Application Publication
(10) Pub. No.: US 2012/0244185 A1
(43) Pub. Date: Sep. 27, 2012

(54) IMMUNIZATION STRATEGY TO PREVENT H1N1 INFECTION

(75) Inventors: Sean N. Tucker, San Francisco, CA (US); Ciaran Scallan, San Francisco, CA (US)

(73) Assignee: Vaxart ,Inc., San Francisco, CA (US)

(21) Appl. No.: 13/496,482

(22) PCT Filed: Sep. 15, 2010
(86) PCT No.: PCT/US2010/048967

§ 371 Date: Jun. 4, 2012

Related U.S. Application Data
(60) Provisional application No. 61/243,070, filed on Sep. 16, 2009.

(51) Int. Cl. A61K 31/7088; A61P 37/04; A61P 31/16; C12N 15/861

(52) U.S. Cl. 424/209.1; 435/320.1

(57) ABSTRACT

The invention provides chimeric viral expression vectors and method of use for providing an immune response against H1N1 influenza. The compositions of the invention can be used as vaccines for H1N1 strains. Advantages include rapid development and availability once a strain is isolated, and effective oral or mucosal administration.

### CROSS-REFERENCES TO RELATED APPLICATIONS

This document claims priority to U.S. Provisional Appl. No. 61/243,070, filed Sep. 16, 2009, the disclosure of which is incorporated by reference in its entirety.

### BACKGROUND OF THE INVENTION

Several different methods have been proposed to prevent influenza infection, but most of these methods use old technologies administered via injection. This can be difficult to accomplish in a pandemic where normal health care has been disrupted. An oral delivery vaccine that can be distributed without qualified medical personnel would have a significant advantage.

The H1N1 pandemic viruses of 2009 target different cell populations than normal influenza. These viruses infect intestinal epithelial cells, as well as lung epithelial cells. Patients infected with H1N1 reported diarrhea and stomach illness along with the usual influenza symptoms.

The present invention provides an oral immunization with substantial benefits in protecting against H1N1 infection. A strong mucosal immune response is generated, including in the intestine, as evidenced by the strong IgA antibody responses against HA in fecal pellets. The oral H1N1 vaccine is also shown to be protective against H1N1 in a direct challenge in vivo.

### BRIEF SUMMARY OF THE INVENTION

The invention provides rapid-response and easily scalable compositions for vaccination against pandemic strains of H1N1 influenza. Upon isolation of the strain, a gene-based viral vaccine according to the invention can be prepared and tested for safety and efficacy within weeks.

Accordingly, in some embodiments, the invention provides chimeric viral expression vectors for protection against H1N1 influenza, said vector comprising an expression cassette comprising (a) a first promoter operably linked to a nucleic acid encoding a toll-like receptor-3 (TLR-3) agonist; and (b) a second promoter operably linked to a nucleic acid encoding a polypeptide from H1N1 influenza. In some embodiments, the viral vector is an adenoviral vector or derived from an adenoviral vector but modified to be attenuated. In some embodiments, the TLR-3 agonist is dsRNA. In some embodiments, the dsRNA is selected from short hairpin RNA (shRNA), viral dsRNA, and siRNA. In some embodiments, the sequence encoding the dsRNA comprises a sequence having substantial identity to a sequence selected from the group consisting of SEQ ID NO:1, SEQ ID NO:2, SEQ ID NO:3, SEQ ID NO:4, SEQ ID NO:5, and SEQ ID NO:6 (SEQ ID NOs:1-6). In some embodiments, the sequence encoding the dsRNA comprises a sequence selected from the group consisting of SEQ ID NOs: 1 -6.

In some embodiments, the polypeptide from H1N1 is a surface antigen of H1N1 or an antigenic fragment thereof. In some embodiments, the H1N1 polypeptide is selected from the group consisting of HA, NA, MP, and NP, and antigenic fragments thereof. In some embodiments, the H1N1 influenza is from the strain A/CA/04/2009.

In some embodiments, the first promoter and the second promoter are the same. In some embodiments, the first promoter and second promoter are different. The promoter can be constitutive or inducible, and can be a viral or mammalian gene promoter, e.g., CMV or SV40.

In some embodiments, the viral expression vector is substantially identical to SEQ ID NO:7, e.g., with at least 90, 92, 95, 96, 98, or 99% identity to SEQ ID NO:7, or to the expression cassette of SEQ ID NO:7 (nucleotide positions 603 to 4194). In some embodiments, the viral expression vector comprises SEQ ID NO:7 or the expression cassette of SEQ ID NO:7. In some embodiments, the viral expression vector is substantially identical to SEQ ID NO:7, but encodes a different H1N1 antigenic polypeptide.

In some embodiments, the invention provides pharmaceutical compositions for protection against H1N1 influenza, said compositions comprising a chimeric viral vector as described herein and a pharmaceutically acceptable carrier. In some embodiments, the pharmaceutical composition is formulated for non-parenteral delivery (administration), e.g., oral, mucosal, or intranasal delivery. In some embodiments, the pharmaceutical composition is formulated for oral delivery.

In some embodiments, the invention provides an oral delivery pharmaceutical composition comprising a chimeric viral vector as described herein and a pharmaceutically acceptable carrier. Thus, in some embodiments, the vector comprises an expression cassette comprising (a) a first promoter operably linked to a nucleic acid encoding a toll-like receptor-3 (TLR-3) agonist; and (b) a second promoter operably linked to a nucleic acid encoding a polypeptide from H1N1 influenza. In some embodiments, the viral vector is an adenoviral vector or derived from an adenoviral vector but modified to be attenuated. In some embodiments, the TLR-3 agonist is dsRNA. In some embodiments, the dsRNA is selected from short hairpin RNA (shRNA), viral dsRNA, and siRNA. In some embodiments, the H1N1 polypeptide is an H1N1 surface antigen. In some embodiments, the H1N1 polypeptide is selected from the group consisting of HA, NA, NP, and MP.

The invention also provides methods for eliciting an immune response to H1N1 influenza, said method comprising administering to a mammal an immunogenically effective amount of a chimeric viral expression vector as described herein, wherein the immune response is directed against the H1N1 influenza polypeptide, and wherein the route of administration is selected from the group consisting of: oral, intranasal, and mucosal. In some embodiments, the administration is oral. In some embodiments, the mammal is a human. In some embodiments, the immune response is selected from the group consisting of a mucosal immune response, a systemic immune response, a humoral immune response, and a cellular immune response. In some embodiments, the immune response is a mucosal immune response. In some embodiments, the immune response can be detected in intestinal mucosa.

### BRIEF DESCRIPTION OF THE DRAWINGS

FIG. 1A. Systemic antibody titers for anti-influenza HA antibodies are comparable at 8 weeks for oral (p.o.) and intramuscular (i.m.) vaccination of DX1. Ad-CA/04/09-Adj was administered orally at two different doses (10⁷ IU or 10⁸ IU) or i.m. at 10⁸ IU. Results from a similar chimeric adenoviral expression vector encoding HA from a non-similar H1N1 are also shown (Ad-PR8-Adj).

FIG. 1B. Results from Hemagglutination Inhibition (HAI) at 3, 5, and 8 weeks post-vaccination confirm similar systemic antibody response to A/CA/04/2009 from oral and i.m. administration.

FIGS. 1C, 1D, and 1F. Oral administration of DX1 vector vaccine provides superior mucosal antibody response compared to i.m. injection. Titers of antibodies specific to HA were measured in intestinal (fecal), vaginal, and lung mucosal tissues, at 8, 10, and 12 weeks post-administration, respectively. Results are shown in panels C, D, and E of FIG. 1. Note that oral administration of DX1 vector provides significantly higher specific IgA titers than high dose i.m. injection. The 10-fold lower oral dose induces an equal or greater IgA response as well. *indicates P<0.05 calculated using Student's T-Test based on OD450 readings.

FIGS. 2A and 2B. Low dose oral administration of DX1 vector provides comparable survival and weight maintenance data to high dose oral and i.m. administration. FIG. 2A shows that 100% of DX1 vaccinated mice receiving a lethal H1N1 challenge survived. Similarly, FIG. 2B shows minimal weight loss in all three groups. Untreated mice, or those vaccinated with adjuvant alone succumbed to the virus soon after challenge. Oral indicates 10⁸ IU DX1, IM indicates 10⁸ IU DX1, lo dose oral indicates 10⁷ IU DX1, Adj control indicates vector expressing dsRNA only.

FIG. 3. Expression of dsRNA adjuvant improves antibody response to the H1N1 HA antigen. Plasma titers of antibodies specific for HA were measured following oral administration of DX1 and DX2 (vector lacking dsRNA encoding sequence).

### DETAILED DESCRIPTION OF THE INVENTION

### I. INTRODUCTION

The present invention provides novel and effective chimeric viral vector vaccines that can be rapidly developed in response to an H1N1 viral pandemic. Once a pandemic strain is isolated, an antigen can be isolated and used to design a vaccine according to the present invention. Animal testing, to determine safety and tolerable dose ranges, as well as efficacy of the vaccine against the particular pandemic strain, can be accomplished on the order of weeks. The chimeric viral vector vaccines comprise a sequence encoding the antigen as well as a sequence encoding a dsRNA adjuvant. The vaccine can be administered non-parenterally and at a low dose to elicit an immune response against the H1N1 antigen. The chimeric viral vectors elicit strong and effective immune responses specific for the H1N1 polypeptide antigen, particularly when administered via a non-parenteral route (e.g., orally, intranasally, or mucosally).

The efficacy of the vector-encoded dsRNA TLR-3 agonist as an adjuvant is also surprising. Use of dsRNA as an adjuvant for viral vectors is counterintuitive considering that the major proposed utility of the dsRNA mimetic poly I:C is as an antiviral agent [Nemes, et al., Proc Soc Exp Biol Med. (1969) 132:776; Schafer, et al, Nature. (1970) 226:449; Fenje, et al, Nature (1970) 226:171.].

### II. DEFINITIONS

The term “chimeric” or “recombinant” as used herein with reference, e.g., to a nucleic acid, protein, or vector, indicates that the nucleic acid, protein or vector, has been modified by the introduction of a heterologous nucleic acid or protein or the alteration of a native nucleic acid or protein. Thus, for example, chimeric and recombinant vectors include nucleic acid sequences that are not found within the native (non-chimeric or non-recombinant) form of the vector. A chimeric viral vector (or chimeric viral expression vector) refers to a viral vector comprising a nucleic acid sequence encoding a heterologous polypeptide.

An “expression vector” or “expression cassette” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter. An expression vector can include more than one expression units or coding sequences, which can be contiguous (e.g., under control of the same promoter) or non-contiguous (e.g., under control of different promoter).

The terms “promoter” and “expression control sequence” refer to nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Promoters include constitutive and inducible promoters. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “TLR-3 agonist” or “Toll-like receptor 3 agonist” as used herein refers to a compound that binds and stimulates the TLR-3. TLR-3 agonists have been identified including double-stranded RNA, virally derived dsRNA, several chemically synthesized analogs to double-stranded RNA including polyinosine-polycytidylic acid (poly I:C)-polyadenylic-polyuridylic acid (poly A:U) and poly I:poly C, and antibodies (or cross-linking of antibodies) to TLR-3 that lead to IFN-beta production [Matsumoto, M, et al, Biochem Biophys Res Commun 24:1364 (2002), de Bouteiller, et al, J Biol Chem 18:38133-45 (2005)]. TLR-3 agonists also include dsRNA encoded by and expressed from a vector, e.g., as described herein.

Double-stranded RNA (dsRNA) is RNA that includes two complementary strands, typically with a complementary stretch of at least 4, 5, 10, 12, 15, 20 or more ribonucleotides. The complementary stretch of a dsRNA can be between 5-10, 5-20, 5-30, 10-20, 10-100, 20-100, and higher. One of skill will appreciate that the complementary stretch need not be 100% complementary for the dsRNA to act as an effective TLR-3 agonist or adjuvant. A dsRNA molecule can comprise an intramolecular double-stranded RNA species (e.g., short hairpin RNA or siRNA) and dsRNA species that comprise at least two separate single-stranded RNA molecules.

Polynucleotides can comprise a designated sequence (i.e., an H1N1 viral sequence that encodes a particular polypeptide or dsRNA or a portion thereof) or a variant of such a sequence that results in an immune response to the desired H1N1 virus or TLR-3 agonism, respectively. Polynucleotide variants may contain one or more substitutions, additions, deletions and/or insertions such that the biological activity of the encoded polypeptide is not diminished, relative to a polypeptide comprising the original sequence. Polynucleotide variants of a given dsRNA can contain one or more substitutions, additions, deletions and/or insertions such that the TLR-3 agonist activity of the encoded dsRNA is not diminished, relative to a dsRNA that does not contain the substitutions, additions, deletions and/or insertions. Variants preferably exhibit at least about 50%, 55%, 60%, 65%, 70%, 75%, 80%, 85%, 90%, 91%, 92%, 93%, 94%, 95%, 96%, 97%, 98%, 99%, to a polynucleotide sequence that encodes a native polypeptide or a portion thereof or to a polynucleotide sequence that encodes a dsRNA with TLR-3 agonist activity.

The term “subject” or “patient,” for the purposes of the present invention, refers to a mammal that can be infected with H1N1 virus. Mammals include humans, as well as non-human mammals such as rodents (e.g., mice and rats), rabbits, livestock animals (e.g., bovine, porcine, and ovine animals) and pets (e.g., dogs and cats), and non-human primates.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “nucleic acid,” “oligonucleotide,” “polynucleotide” and like terms are used interchangeably herein to refer to polymers of deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

Antigen—refers to a protein or part of a polypeptide chain that can be recognized by T cell receptors and/or antibodies. Typically, antigens are derived from bacterial, viral, or fungal proteins, or from abnormal proteins expressed by cancer cells.

An “immunogenically effective dose or amount” of a composition of the present invention is an amount that elicits or modulates an immune response specific for the desired antigenic polypeptide. Immune responses include humoral immune responses and cell-mediated immune responses. An immunogenic composition can be used therapeutically or prophylactically to treat or prevent H1N1 infection.

The terms “dose” and “dosage” are used interchangeably herein. A dose refers to the amount of active ingredient given to an individual at each administration. The dose for the purposes of the present invention can be expressed in a number of ways, e.g., mg/mL or IU of the viral vector, mg or IU per kg or lb body weight of the subject, or in terms of the amount of antigenic polypeptide produced by the viral vector. The dose will vary depending on a number of factors, including frequency of administration; size and tolerance of the individual; severity of the H1N1 outbreak; risk of infection; and the route of administration. One of skill can determine how to modify a minimal dose depending on the above factors. The initial dose can be used as a baseline, and modified based on the reaction of the individual.

A “dosage form” refers to the particular format of the pharmaceutical, and depends on the route of administration. For example, a dosage form can be in a tablet or liquid, e.g., for oral delivery, a suppository, e.g., for rectal or vaginal administration, or a liquid for injection. A “single dosage form” refers both to the format of the pharmaceutical composition and the amount of active ingredient as discussed above.

“Protection” or “prevention” of infection refers to reducing the potential that an individual will develop H1N1 infection, e.g., detected by observing symptoms, or by detecting viral or antibody titer. The term includes protection from further infection or worsening infection, e.g., in the case of an individual that is already suffering from an infection, e.g., from a different strain of H1N1 or a different virus. The potential for an infection can be reduced to such an extent that the individual does not display symptoms upon exposure to the infectious agent. For example, protecting an individual infection can refer to the ability of a chimeric viral vector of the present invention to prevent or reduce infection from occurring or recurring.

“Treating” or “reducing” H1N1 infection refers to reducing the amount of the infective agent (e.g., number of cells or viral particles) in the individual, reducing the severity of symptoms, and/or reducing the frequency of symptoms. In some embodiments, the number of infective agents is reduced by at least 10%, as compared, e.g., to the individual before administration or to a control individual not undergoing treatment. In some embodiments the number of infective agents is reduced by at least 25%, 50%, 75%, 80%, or 90%. In some embodiments, the infection is no longer detectable, e.g., by symptoms or general diagnostic techniques.

One of skill will appreciate that the terms protecting, preventing, and treatment are not absolute terms, but a general improvement of outcome compared to untreated or differently treated controls. For example, the compositions of the invention are considered protective if a treated individual or population is less likely to develop H1N1 infection than an individual or population that is not treated according to the invention, but exposed to similar conditions.

The term “infection” for the purposes of the invention refers to the presence of a potentially pathogenic H1N1 influenza strain in an individual. Infection does not necessarily imply that an infected individual will experience symptoms from the infective agent. H1N1 infection typically occurs via mucosal tissues, e.g., epithelial linings of the reproductive tract, gastrointestinal tract, oral and esophageal surfaces, nasal cavity, and bronchial lining.

An agent “interferes with” or “protects from” infection when it reduces the ability of a targeted H1N1 strain to colonize the mucosal surface or penetrate the mucosal barrier, e.g., to infect cells of the individual. For example, an agent interferes with infection if fewer cells are exposed to or infected by the virus. Reduction can be determined by comparison, e.g., to the individual before treatment or to a control individual exposed to similar conditions but not undergoing treatment.

The term “isolated” is not an absolute term, but refers to a material that is substantially free of contaminants or components that accompany the material in its native state.

“Humoral immune responses” are mediated by cell free components of the blood, i.e., plasma or serum, e.g., antibodies. Transfer of the serum or plasma from one individual to another transfers humoral immunity.

“Cell mediated immune responses” are mediated by antigen specific lymphocytes, e.g., cytotoxic T cells (CTLs). Transfer of the antigen specific lymphocytes from one individual to another transfers immunity.

A “therapeutic dose” or “therapeutically effective amount” or “effective amount” of a chimeric viral vector or a composition comprising a chimeric viral vector is an amount of the vector or composition comprising the vector which prevents, alleviates, abates, or reduces the severity of symptoms of the targeted H1N1 virus.

Antibody refers to a polypeptide encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

T cells—refer to a particular class of lymphocytes that express a specific receptor (T cell receptor) encoded by a family of genes. The recognized T cell receptor genes include alpha, beta, delta, and gamma loci, and the T cell receptors typically (but not universally) recognize a combination of MHC plus a short peptide.

An adaptive immune response refers to specific recognition of an antigen by T cells (through the T cell receptor (TCR)) or B cells (through the BCR or antibody/immunoglobulin).

Antigen presenting cells (APCs) present immunogenic peptides or fragments thereof to T cells to activate or enhance an immune response. APCs include dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may be isolated from any of a variety of biological fluids and organs including bone marrow, peripheral blood, tumor and peritumoral tissues, and may be autologous, allogeneic, syngeneic or xenogeneic cells. APCs typically utilize a receptor from the major histocompatability (MHC) locus to present short polypeptides to T cells.

Adjuvants are known in the art, and refer to non-specific immune response enhancers. Suitable adjuvants include, e.g., dsRNA and dsRNA mimetics, cholera toxin, monophosphoryl lipid A (MPL), Freund's Complete Adjuvant, Freund's Incomplete Adjuvant, Quil A, and Al(OH). Adjuvants can also be those substances that cause APC activation and enhanced presentation of T cells through secondary signaling molecules like Toll-like receptors. Examples of Toll-like receptors include the receptors that recognize double-stranded RNA, bacterial flagella, LPS, CpG DNA, and bacterial lipopeptide (reviewed in Abreu et al., J Immunol, 174(8), 4453-4460 (2005)).

The terms “polypeptide,” “peptide” and “protein” refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine I, Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The phrase “selectively (or specifically) hybridizes to” refers to the binding, duplexing, or hybridizing of a polynucleotide only to a particular nucleotide sequence, i.e., according to Watson-Crick complementarity.

Nucleic acids that do not hybridize to each other under given conditions are still considered substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to fusion proteins can be selected to obtain only those polyclonal antibodies that are specifically immunoreactive with fusion protein and not with individual components of the fusion proteins. This selection may be achieved by subtracting out antibodies that cross-react with the individual antigens.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The terms “identical” or percent “identity,” in the context of two or more nucleic acids (e.g., a dsRNA or antigen encoding sequence) or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%, or more identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 10 to about 100, about 20 to about 75, about 30 to about 50 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions from about 10 to about 500, about 25 to about 200, 50 to about 150, in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An example of an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (at the website for NCBI, ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

### III. COMPOSITIONS OF THE INVENTION

The invention provides compositions comprising chimerical viral vectors. In some embodiments, the chimeric viral vectors of the invention comprise a first promoter operably linked to a nucleic acid encoding an antigenic H1N1 polypeptide and a second promoter operably linked to a nucleic acid encoding a TLR3 agonist. The first and second promoters may be the same or different, inducible, constitutive, or tissue-specific. In some embodiments, the first and second promoters are independently selected from: the beta actin promoter and the CMV promoter.

In some embodiments, the chimeric viral vector comprises an adenoviral genome (minus the E1 and E3 genes) and a nucleic acid encoding a dsRNA. The chimeric vector can be administered to a cell that expresses the Ad E1 gene such that recombinant adenovirus (rAd) is produced by the cell. This rAd can be harvested and is capable of a single round of infection that will deliver the transgenic composition to another cell within a mammal in order to elicit immune responses to the antigenic polypeptide.

A. Suitable Viral Vectors

Any viral expression vector can be used for the present invention. Viral vaccines are typically attenuated (e.g., replication incompetent). Suitable viral expression vectors can derived from adenoviruses, modified vaccinia ankara (MVA; see, e.g., Kumar and Seth (2004) Gene Ther. Mol. Biol. 8:193-200), vaccinia viruses, herpes viruses, baculoviruses, adeno-associated viruses (AAV), and rubella virus (e.g., as described in Pugachev et al. (2000) J. Virology 74:10811-15). In some embodiments, the vector is an adenoviral vector, e.g., derived from adenovirus 5, for example, Ad5 with deletions of the E1/E3 regions and Ad5 with a deletion of the E4 region. Other suitable adenoviral vectors include strains 2, orally tested strains 4 and 7, enteric adenoviruses 40 and 41, and other strains (e.g. Ad34) that are sufficient for delivering an antigen and eliciting an adaptive immune response to the transgene antigen [Lubeck et al., Proc Natl Acad Sci USA, 86(17), 6763-6767 (1989); Shen et al., J Virol, 75(9), 4297-4307 (2001); Bailey et al., Virology, 202(2), 695-706 (1994)]. In some embodiments, the adenoviral vector is a live, replication incompetent adenoviral vector (such as E1 and E3 deleted rAd5), live and attenuated adenoviral vector (such as the E1B55K deletion viruses), or a live adenoviral vector with wild-type replication.

The transcriptional and translational control sequences in expression vectors to be used in transforming vertebrate cells in vivo may be provided by viral sources. For example, commonly used promoters and enhancers are derived, e.g., from beta actin, adenovirus, simian virus (SV40), and human cytomegalovirus (CMV). For example, vectors allowing expression of proteins under the direction of the CMV promoter, SV40 early promoter, SV40 later promoter, metallothionein promoter, murine mammary tumor virus promoter, Rous sarcoma virus promoter, transducer promoter, or other promoters shown effective for expression in mammalian cells are suitable. Further viral genomic promoters, control and/or signal sequences can be used, provided such control sequences are compatible with the host cell chosen.

B. H1N1 Polypeptides

Nucleic acids encoding suitable heterologous polypeptides may be derived from H1N1 influenza antigens. H1N1 infects mucosal tissues, such as intestine, thus antigens that give rise to a robust mucosal response are of particular interest. Antigenic polypeptides include H1N1 viral surface antigens and fragments thereof. Antigenic polypeptides include, e.g., HA, NA, MP, and NP from H1N1 influenza.

One of skill in the art will understand how to identify and isolate antigenic polypeptides from a newly isolated or particularly virulent H1N1 strain, e.g., using comparisons to known influenza sequences.

A recombinant expression vector can include nucleotide sequences encoding an immunogenic polypeptide operably linked to suitable transcriptional or translational regulatory elements derived from mammalian or viral genes. Such regulatory elements include promoters, a sequence encoding suitable mRNA ribosomal binding sites, and sequences which control the termination of transcription and translation. An origin of replication, a selectable marker to facilitate recognition of transformants, and/or an operator sequence to control transcription may additionally be incorporated. In some embodiments, the components of the recombinant expression vector can be divided between more than one virus such that the gene products are on two different vectors, and the vectors are used for co-transduction to provide all the gene products in trans. There may be reasons to divide up the gene products such as size limitations for insertions, or toxicity of the combined gene products to the virus produce cell-lines.

C. TLR-3 Agonists

TLR-3 agonists are used to enhance the immune response to the H1N1 polypeptide. The TLR-3 agonist can be delivered simultaneously on the same expression vector encoding an antigen of interest. In some embodiments, the TLR-3 agonist can be delivered separately (i.e., temporally or spatially) from the expression vector encoding an antigen of interest. For example, the expression vector may be administered via a non-parenteral route (e.g., orally, intranasally, or mucosally), while the TLR-agonist is delivered by a parenteral route (e.g., intramuscularly, intraperitoneally, or subcutaneously).

TLR-3 agonists include, for example, short hairpin RNA, virally derived RNA, short segments of RNA that can form double-strands, and short interfering RNA (siRNA). In some embodiments of the invention, the TLR-3 agonist is virally derived dsRNA, such as for example, a dsRNA derived from a Sindbis virus or dsRNA viral intermediates [Alexopoulou et al, Nature 413:732-8 (2001)].

In some embodiments, the TLR-3 agonists is a short hairpin RNA. Short hairpin RNA sequences typically comprise two complementary sequences joined by a linker sequence. The particular linker sequence is not a critical aspect of the invention. Any appropriate linker sequence can be used so long as it does not interfere with the hybridization of the two complementary sequences to form a dsRNA. Similarly, the sequence of the complementary stretches is not necessarily critical, as long as the strands form a double-stranded molecule. The strands need not be 100% complementary as long as the double-stranded structure is maintained. The complementary stretch is typically in the range of 10-30 nucleotides, e.g., 4-10, 5-15, 5-20, 10-25, 20-30, or 10-30 nucleotides, but can be longer, e.g., 20-100 nucleotides in length.

In some embodiments, the sequence encoding the short hairpin RNA comprises a sequence set forth in any one of SEQ ID NOs:1-6, a sequence with substantial identity to a sequence set forth in SEQ ID NOs:1-6, or a variant of a sequence set forth in SEQ ID NOs:1-6. The dsRNA that is a TLR-3 agonist does not encode a particular polypeptide, but results in production of a pro-inflammatory cytokine (e.g. IL-6, IL-8, TNF-alpha, IFN-alpha, IFN-beta) when contacted with a responder cell (e.g., a dendritic cell, a peripheral blood mononuclear cell, or a macrophage) in vitro or in-vivo.

In some embodiments, the nucleic acid encoding the TLR-3 agonist (e.g., an expressed dsRNA) and the chimeric viral vector comprising the nucleic acid encoding an H1N1 antigen are administered in the same formulation. In other cases, the nucleic acid encoding the TLR-3 agonist and the chimeric viral vector comprising the nucleic acid encoding the H1N1 antigen are administered in different formulations, either simultaneously or sequentially.

In some embodiments, the nucleic acid encoding the TLR-3 agonist and the nucleic acid encoding the H1N1 antigen are under the control of the same promoter. In other embodiments, the nucleic acid encoding the TLR-3 agonist and the nucleic acid encoding the H1N1 antigen are under the control of different promoters.

Several chemically synthesized analogs to double-stranded RNA are commercially available. These include polyinosine-polycytidylic acid (poly I:C), polyadenylic:polyuridylic acid (poly A:U), and poly I:poly C. Antibodies (or cross-linking of antibodies) to TLR-3 can also lead to IFN-beta or pro-inflammatory cytokine production [Matsumoto et al, Biochem. Biophys. Res. Commun. 24:1364 (2002), de Bouteiller et al, J Biol. Chem. 18:38133-45 (2005)]. Commercially available siRNA segments of any sequence can also be obtained through sources such as Invitrogen.

### IV. PHARMACEUTICAL COMPOSITIONS

Pharmaceutical compositions comprising the vectors described herein can include pharmaceutically acceptable carriers, which may be biologically active or inactive. Pharmaceutical compositions can generally be used for prophylactic and therapeutic purposes. In some embodiments, the pharmaceutical composition can be designed to protect against stomach degradation. For the oral environment, several such compositions are available including the Eudragit and the TimeClock release systems as well as other methods specifically designed for adenovirus [Lubeck et al., Proc Natl Acad Sci USA, 86(17), 6763-6767 (1989); Chourasia and Jain, J Pharm Pharm Sci, 6(1), 33-66 (2003)]. There are also several methods already described for microencapsulation of DNA and drugs for oral delivery (see, e.g., U.S. Patent Publication No. 2004043952). In some embodiments, the Eudragit system can be used to deliver the chimeric viral vector to the lower small intestine. However, delivery to other locations of the small intestine is also advantageous for protection against H1N1.

The chimeric viral vectors on the invention can be delivered using any delivery systems known in the art. Numerous gene delivery techniques are well known, such as those described by Rolland (1998) Crit. Rev. Therap. Drug Carrier Systems 15:143-198, and references cited therein.

It will be apparent that an immunogenic composition can contain pharmaceutically acceptable salts of the polynucleotides encoding the H1N1 immunogenic polypeptides. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts). Some particular examples of salts include phosphate buffered saline and saline for injection.

Any suitable carrier known to those of ordinary skill in the art may be employed in the pharmaceutical compositions of this invention. Suitable carriers include, for example, water, saline, alcohol, a fat, a wax, a buffer, a solid carrier, such as mannitol, lactose, starch, magnesium stearate, sodium saccharine, talcum, cellulose, glucose, sucrose, and magnesium carbonate, or biodegradable microspheres (e.g., polylactate polyglycolate). Suitable biodegradable microspheres are disclosed, for example, in U.S. Pat. Nos. 4,897,268; 5,075,109; 5,928,647; 5,811,128; 5,820,883. The chimeric viral vector can be encapsulated within the biodegradable microsphere or associated with the surface of the microsphere.

Such compositions can comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention can be formulated as a lyophilizate. Compounds can also be encapsulated within liposomes using well known technology.

In some embodiments, the compositions can comprise an additional adjuvant. Suitable adjuvants include, for example, the lipids and non-lipid compounds, cholera toxin (CT), CT subunit B, CT derivative CTK63, E. coli heat labile enterotoxin (LT), LT derivative LTK63, Al(OH)₃, and polyionic organic acids as described in e.g., WO 04/020592, Anderson and Crowle, Infect. Immun. 31(1):413-418 (1981), Roterman et al., J. Physiol. Pharmacol., 44(3):213-32 (1993), Arora and Crowle, J. Reticuloendothel. 24(3):271-86 (1978), and Crowle and May, Infect. Immun. 38(3):932-7 (1982)). Suitable polyionic organic acids include for example, 6,6′-[3,3′-demithyl[1,1′-biphenyl]-4,4′-diyl]bis(azo)bis[4-amino-5-hydroxy-1,3-naphthalene-disulfonic acid] (Evans Blue) and 3,3′-[1,1′biphenyl]-4,4′-diylbis(azo)bis[4-amino-1-naphthalenesulfonic acid] (Congo Red). It will be appreciated by those of skill in the art that the polyionic organic acids can be used for gene-based vaccination methods in conjunction with any route of administration.

Other suitable adjuvants include topical immunomodulators such as members of the imidazoquinoline family, e.g., imiquimod and resiquimod (see, e.g., Hengge et al., Lancet Infect. Dis. 1(3):189-98 (2001).

Additional suitable adjuvants are commercially available as, for example, additional alum-based adjuvants (e.g., Alhydrogel, Rehydragel, aluminum phosphate, Algammulin); oil based adjuvants (Freund's Incomplete Adjuvant and Complete Adjuvant (Difco Laboratories, Detroit, Mich.), Specol, RIBI, TiterMax, Montanide ISA50 or Seppic MONTANIDE ISA 720); nonionic block copolymer-based adjuvants, cytokines (e.g., GM-CSF or Flat3-ligand); Merck Adjuvant 65 (Merck and Company, Inc., Rahway, N.J.); AS-2 (SmithKline Beecham, Philadelphia, Pa.); salts of calcium, iron or zinc; an insoluble suspension of acylated tyrosine; acylated sugars; cationically or anionically derivatized polysaccharides; polyphosphazenes; biodegradable microspheres; monophosphoryl lipid A and Quil A. Cytokines, such as GM-CSF or interleukin-2, -7, or -12, are also suitable adjuvants. Hemocyanins (e.g., keyhole limpet hemocyanin) and hemoerythrins can also be used. Polysaccharide adjuvants such as, for example, chitin, chitosan, and deacetylated chitin are also suitable as adjuvants. Other suitable adjuvants include muramyl dipeptide (MDP, N acetylmuramyl L alanyl D isoglutamine) bacterial peptidoglycans and their derivatives (e.g., threonyl-MDP, and MTPPE). BCG and BCG cell wall skeleton (CWS) can also be used as adjuvants, with or without trehalose dimycolate. Trehalose dimycolate can also be used (see, e.g., U.S. Pat. No. 4,579,945). Detoxified endotoxins are also useful as adjuvants alone or in combination with other adjuvants (see, e.g., U.S. Pat. Nos. 4,866,034; 4,435,386; 4,505,899; 4,436,727; 4,436,728; 4,505,900; and 4,520,019. The saponins QS21, QS17, QS7 are also useful as adjuvants (see, e.g., U.S. Pat. No. 5,057,540; EP 0362 279; WO 96/33739; and WO 96/11711). Other suitable adjuvants include Montanide ISA 720 (Seppic, France), SAF (Chiron, Calif., United States), ISCOMS (CSL), MF-59 (Chiron), the SBAS series of adjuvants (e.g., SBAS-2, SBAS-4 or SBAS-6 or variants thereof, available from SmithKline Beecham, Rixensart, Belgium), Detox (Corixa, Hamilton, Mont.), and RC-529 (Corixa, Hamilton, Mont.).

Superantigens are also contemplated for use as adjuvants. Superantigens include Staphylococcus exoproteins, such as the α, β, γ and Δ enterotoxins from S. aureus and S. epidermidis, and the α, β, γ and Δ E. coli exotoxins. Common Staphylococcus enterotoxins are known as staphylococcal enterotoxin A (SEA) and staphylococcal enterotoxin B (SEB), with enterotoxins through E (SEE) being described (Rott et al., 1992). Streptococcus pyogenes B (SEB), Clostridium perfringens enterotoxin (Bowness et al., 1992), cytoplasmic membrane-associated protein (CAP) from S. pyogenes (Sato et al., 1994) and toxic shock syndrome toxin 1 (TSST 1) from S. aureus (Schwab et al., 1993) are further useful superantigens.

Within the pharmaceutical compositions provided herein, the adjuvant composition can be designed to induce, e.g., an immune response predominantly of the Th1 or Th2 type. High levels of Th1-type cytokines (e.g., IFN-gamma, TNF-alpha, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Oral delivery of a composition comprising an immunogenic H1N1 polypeptide can induce an immune response that includes Th1- and Th2-type responses.

The compositions described herein may be administered as part of a sustained release formulation (i.e., a formulation such as a capsule or sponge that effects a slow release of compound following administration). Such formulations may generally be prepared using well known technology (see, e.g., Coombes et al. (1996) Vaccine 14:1429-1438). Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound (see, e.g., WO 94/20078; WO 94/23701; and WO 96/06638). The amount of active compound contained within a sustained release formulation depends upon the site of implantation, the rate and expected duration of release and the nature of the condition to be treated or prevented.

The pharmaceutical composition can be presented in unit-dose or multi-dose containers, such as sealed ampoules or vials. Such containers are preferably hermetically sealed to preserve sterility of the formulation until use. In general, formulations can be stored as suspensions, solutions or emulsions in oily or aqueous vehicles. Alternatively, a pharmaceutical composition may be stored in a freeze-dried condition requiring only the addition of a sterile liquid carrier immediately prior to use.

### V. THERAPEUTIC USES OF THE INVENTION

The invention provides vaccines to reduce the likelihood or severity of H1N1 infection. As used herein, a “subject” or a “patient” refers to any warm-blooded animal, such as, for example, a rodent, a feline, a canine, or a primate, e.g., human.

The immunogenic compositions may be used to treat at any stage, e.g., to prevent infection with a different strain of H1N1 in an individual that has already experienced infection with an H1N1 strain. Within such methods, pharmaceutical compositions are typically administered to a patient. The patient may or may not yet be infected or exposed. Accordingly, the above pharmaceutical compositions may be used to prevent the development of infection or symptoms. Infection or exposure can be diagnosed according to methods known in the art, e.g., by measuring viral titer or viral-specific antibody in a sample from the patient.

Immunotherapy is typically active immunotherapy, in which treatment relies on the in vivo stimulation of the endogenous host immune system to react against virus or virally infected cells.

Frequency of administration of the prophylactic or therapeutic compositions described herein, as well as dosage, will vary from individual to individual, and may be readily established using standard techniques. For example, between 1 and 10 doses can be administered over a set period of time (e.g., 1 or 2 weeks, or 1, 2, or 3 months) or over the course of a pandemic outbreak. In some embodiments, 3 doses are administered, at intervals of 1 month. In some embodiments, 2-3 doses are administered every 2-3 months. Booster vaccinations can be given periodically thereafter, e.g., for the duration of the pandemic, or if there is a risk of reemergenc of the targeted strain or a similar strain.

One of skill in medicine will appreciate that dosage and frequency of administration will vary for individual patients. A suitable dose is an amount of the composition that, when administered as described above, is capable of promoting an anti-viral immune response that is at least 10-50% above the basal (i.e., untreated) level. Such response can be monitored by measuring the specific antibodies in a patient or by vaccine-dependent generation of cytolytic T cells capable of killing virally infected cells in vitro. Such vaccines should also be capable of causing an immune response that leads to an improved clinical outcome in vaccinated patients as compared to non-vaccinated patients.

In general, an appropriate dosage and treatment regimen provides the active compound(s) in an amount sufficient to provide therapeutic and/or prophylactic benefit. Such a response can be monitored by establishing an improved clinical outcome (e.g., reduced severity of symptoms such as weight loss, no or low detectable viral titer, greater chance of survival) in treated patients as compared to non-treated patients. Such immune responses can generally be evaluated using standard proliferation, cytotoxicity or cytokine assays described above. Immune responses can be determined using samples obtained from a patient before and after treatment, or compared to an averaged response of a population, e.g., of similar individuals.

Detection of immunocomplexes formed between immunogenic polypeptides and antibodies in body fluid can be used to monitor the effectiveness of therapy. Samples of body fluid taken from an individual prior to and subsequent to vaccination can be analyzed for the immunocomplexes by the methodologies described above. A substantial change in the number of immunocomplexes in the second sample (post-vaccination) relative to the first sample (pre-vaccination) can reflect successful vaccination.

A. Administration of the Compositions of the Invention

A composition comprising the chimeric viral vector can be administered by any non-parenteral route (e.g., orally, intranasally, or mucosally via, for example, the vagina, lungs, salivary glands, nasal cavities, small intestine, colon, rectum, tonsils, or Peyer's patches). The composition can be administered alone or with an additional adjuvant as described above.

B. Detection of an Immune Response to Antigens of Interest

An immune response to the H1N1 antigenic polypeptide can be detected using any means know in the art including, for example detecting specific activation of CD4⁺ or CD8⁺ T cells or by detecting the presence of antibodies that specifically bind to the polypeptide.

Specific activation of CD4⁺ or CD8⁺ T cells associated with a mucosal, humoral, or cell-mediated immune response may be detected in a variety of ways. Methods for detecting specific T cell activation include, but are not limited to, detecting the proliferation of T cells, the production of cytokines (e.g., lymphokines), or the generation of cytolytic activity (i.e., generation of cytotoxic T cells specific for the immunogenic polypeptide). For CD4⁺ T cells, activation can be determined by detecting proliferation. For CD8⁺ T cells, activation can be detected by the generation of cytolytic activity using ⁵¹Cr release assays (see, e.g., Brossart and Bevan, Blood 90(4): 1594-1599 (1997) and Lenz et al., J. Exp. Med. 192(8):1135-1142 (2000)).

Detection of the proliferation of T cells may be accomplished by a variety of known techniques. For example, T cell proliferation can be detected by measuring the rate of DNA synthesis. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca2+ flux, or dye uptake, such as 3-(4,5-dimethylthiazol-2-yl)-2,5-diphenyltetrazolium. Alternatively, synthesis of lymphokines (e.g., interferon-gamma) can be measured or the relative number of T cells that can respond to the immunogenic polypeptide may be quantified.

Antibody immune responses (humoral immune responses or B cell responses), including mucosal antibody responses can be detected using immunoassays known in the art [Tucker et al., Mol Therapy, 8, 392-399 (2003); Tucker et al., Vaccine, 22, 2500-2504 (2004)]. Suitable immunoassays include the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “western blot” method of Gordon et al. (U.S. Pat. No. 4,452,901); immunoprecipitation of labeled ligand (Brown et al. (1980) J. Biol. Chem. 255:4980-4983); enzyme-linked immunosorbent assays (ELISA) as described, for example, by Raines et al. (1982) J. Biol. Chem. 257:5154-5160; immunocytochemical techniques, including the use of fluorochromes (Brooks et al. (1980) Clin. Exp. Immunol. 39:477); and neutralization of activity (Bowen-Pope et al. (1984) Proc. Natl. Acad. Sci. USA 81:2396-2400). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876.

The examples are provided to illustrate the invention but not to limit its scope. Other variants of the invention will be readily apparent to one of ordinary skill in the art and are encompassed by the appended claims. All publications, databases, patents, patent applications, and accession nos. cited herein are hereby incorporated by reference in their entirety for all purposes.

### IV. EXAMPLES

### Example 1

### Construction of a Chimeric Adenoviral Vector (DX1) Encoding a dsRNA Adjuvant and an H1N1 HA Polypeptide

The coding sequence for HA from influenza strain A/CA/04/09 (synthesized by Blue Heron) was cloned into a KpnI restriction site of a shuttle vector (pShuttleCMV, Qbiogene). The start codon for the HA sequence is immediately downstream of a CMV promoter with a small intron. A bGH poly A sequence immediately follows the HA gene. The pShuttle vector also includes a sequence encoding a dsRNA hairpin adjuvant (luc1, SEQ ID NO:1), so that both antigen and adjuvant are both encoded on the same vector but with separate CMV promoters. This shuttle vector is called pCMV-HA-CMV-luc1.

Homologous recombination of pCMV-HA-CMV-luc1 with the adenoviral backbone, pAd (Qbiogene), was performed in order to generate a plasmid construct capable of producing a recombinant Adenoviral vector (E1/E3 deleted) that expressed the HA transgene and the sequence coding for the dsRNA hairpin luc1. This vector is called pAd-DX1, and shown in SEQ ID NO:7. Recombinant Ad was generated by transfecting a linerized pAd-DX1 expression construct into HEK293 cells. This viral vector is called DX1. Viral titers (infectious units (IU/ml) were measured by a hexon immunostaining assay which is similar to the QuickTiter™ Immunoassay described by Cell Biolabs Inc. (San Diego, Calif.)

### Example 2

### Oral DX1 is Superior to Injected rAdS for Inducing an Antigen Specific Immune Responses in Mucosal Tissues

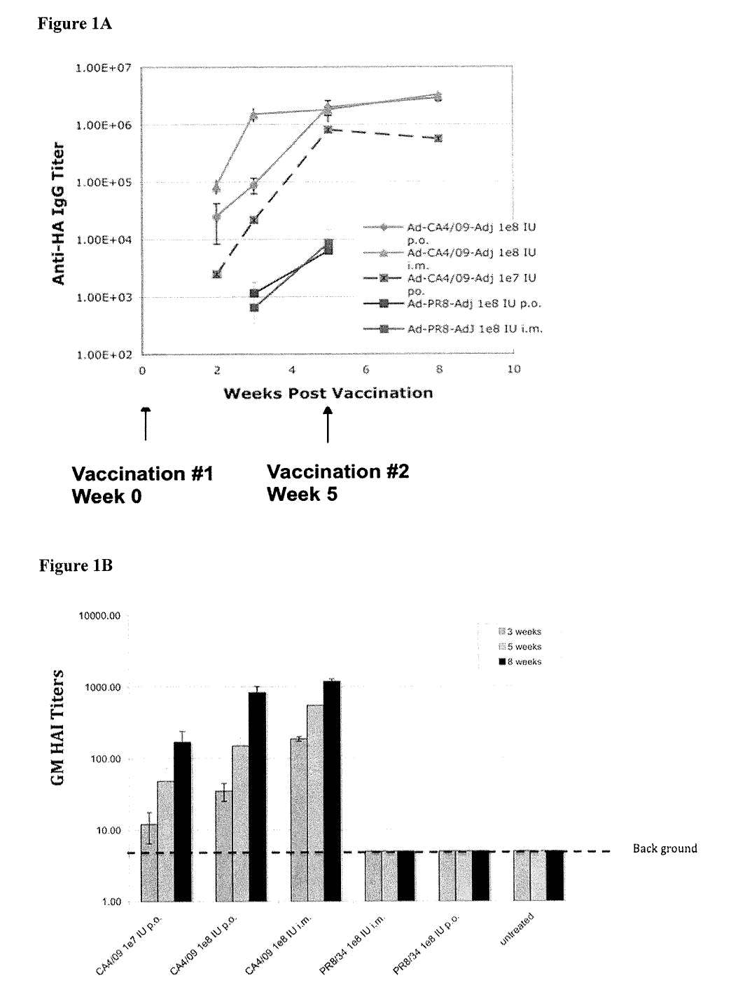

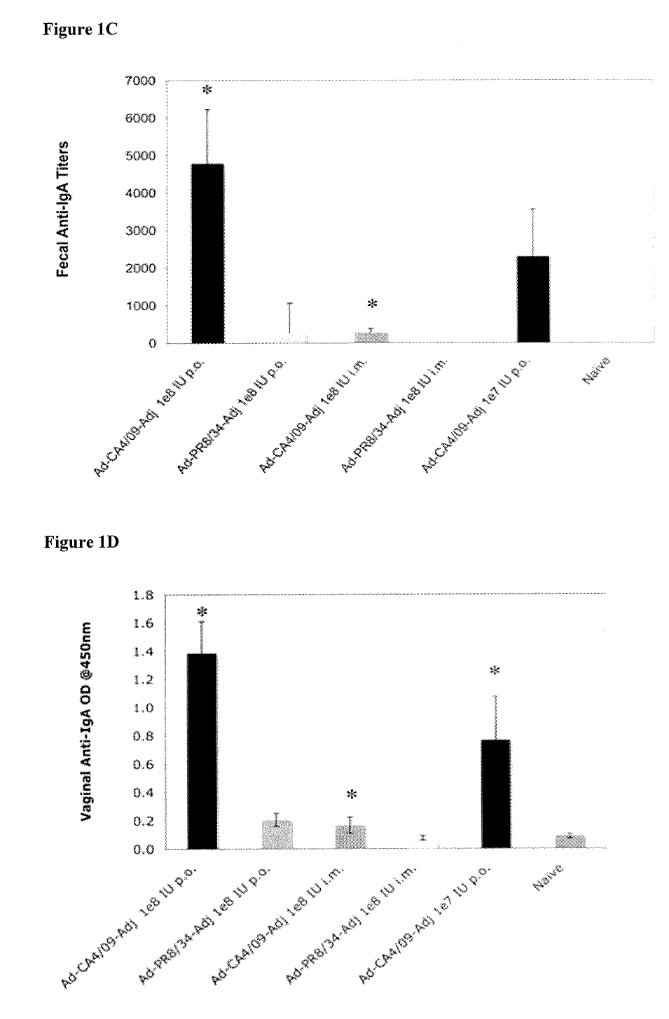

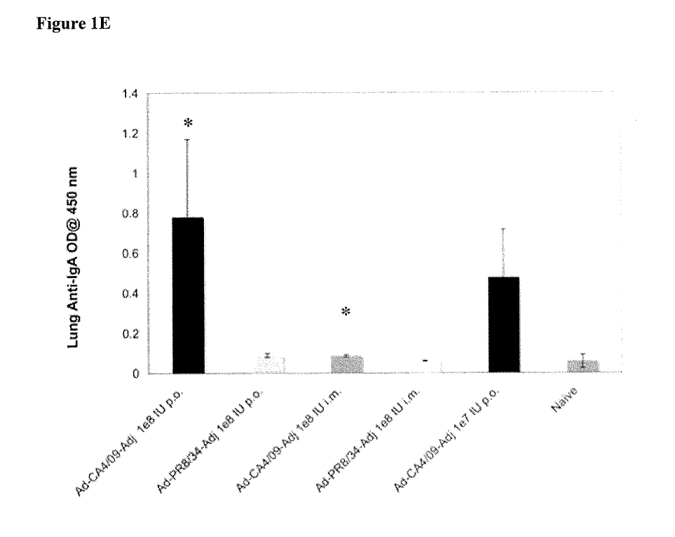

Generating a high antibody titer to an orally-administered, gene-based antigen delivery poses a challenge. We sought to determine whether the DX1 vector could induce substantial systemic and mucosal antibody titers. As explained above, DX1 expresses HA under control of the CMV promoter in an E1/E3 deleted adenovirus type 5 vector. Balb/c female mice were administered DX1 vector by oral gavage on weeks 0 and 5 with 1.0×10⁷ IU or 1.0×10⁸ IU DX1 (labeled Ad-CA04/09-Adj in FIG. 1) or by intramuscular (i.m.) immunization of 1.0×10⁸ IU of DX1 (N=6 for each group). Control groups were vaccinated with a similar Ad vector expressing an H1N1 HA antigen from influenza lab strain A/PR/8/34 (labeled Ad-PR8/34-Adj in FIG. 1). A/PR/8/34 is not considered highly similar to the A/CA/04/09 H1N1 strain. The percent amino acide identity of the HA protein is on the order of 75-85%. The control Ad vector was also administered either orally (labeled p.o.) or by i.m. injection. Untreated mice provided an additional control.

To determine systemic antibody response, we measured antibody titers in plasma post-vaccination. Antibody titers to A/CA/4/09 HA were measured in the plasma 2, 3, 5 and 8 weeks after the initial virus administration by anti-HA IgG ELISA (FIG. 1A). Oral and i.m. immunizations with DX1 produced comparable systemic anti-HA IgG immunity by 8 weeks post vaccination.

Hemagglutinination inhibition (HAI) antibody titers were also measured in plasma at 3, 5, and 8 weeks post-vaccinataion (FIG. 1B). Significant HAI titers were elicitated in both oral (p.o.) groups. By 8 weeks, the high oral dose showed essentially equivalent HAI titer as that elicited by muscle injection. The PR/8/34 HA vector did not induce HAI titers above the background of the assay, as the ELISA relied on antibody binding to the HA antigen from A/CA/4/09.

Mucosal IgA antibody titers to HA were measured in fecal samples at week 8 post-administration (FIG. 1C), vaginal lavages at week 10 post-administration (FIG. 1D), and lung lavages at week 12 post-administration (FIG. 1E). These results demonstrate that oral delivery of DX1 vector performed significantly better than i.m. delivery in eliciting antibody responses to the protein HA in both local (intestine) and distal (vagina & lungs) mucosal tissues. In particular, the average antibody titer to HA was 17-fold better in intestinal tissues (fecal IgA) after oral immunizations than after i.m. vaccination (FIG. 1C).

Surprisingly, a 10-fold lower dose of orally administered DX1 induced an equivalent or better specific IgA response than i.m. delivery of DX1 in lung washes, vaginal washes, and fecal pellets (FIGS. 1C, 1D, 1E). In addition, oral administration of the poorly cross-reactive

PR/8/34 vector resulted in a specific IgA response similar to that of the i.m. administered DX1. The specific IgA results indicate that the oral use of DX1 provides superior protection against H1N1 influenza, a mucosally invading pathogen. Thus, use of the present adenoviral vaccine approach can allow for lower effective vaccine doses. In addition, vaccination with a mismatched antigen can still impart some specific immune protection. These are important considerations during a pandemic, when vaccine supply is critical and the availability of a completely matched vaccine strain may not be possible.

### Example 3

### Demonstration that Oral Delivery of DX1 can Protect Animals Against influenza Challenge

Mice were immunized with the groups listed in Table 1 on weeks 0 and 4. Oral immunization was performed by oral gavage with either 1×10⁸ or 1×10⁷ IU of DX1 (Groups A and C). In order to control for delivery, an i.m. immunization with DX1 was also performed (Group B). To control for possible downstream effects of the adjuvant, we used an adjuvant control vector that expresses the dsRNA adjuvant, but not the antigen. Untreated mice were used as a negative control. The number of animals in each challenge group was 6.

The animals were challenged with a pandemic H1N1 virus (A/CA/07/2009) on week 9 (day 64), using a dose of 8.6×10⁵ TCID₅₀. The A/CA/07/2009 is considered very similar to A/CA/04/09, and the HA proteins share greater than 95% amino acid identity. Animals were measured for weight loss over 14 days post challenge as weight loss is an indicator of the health of the animals.

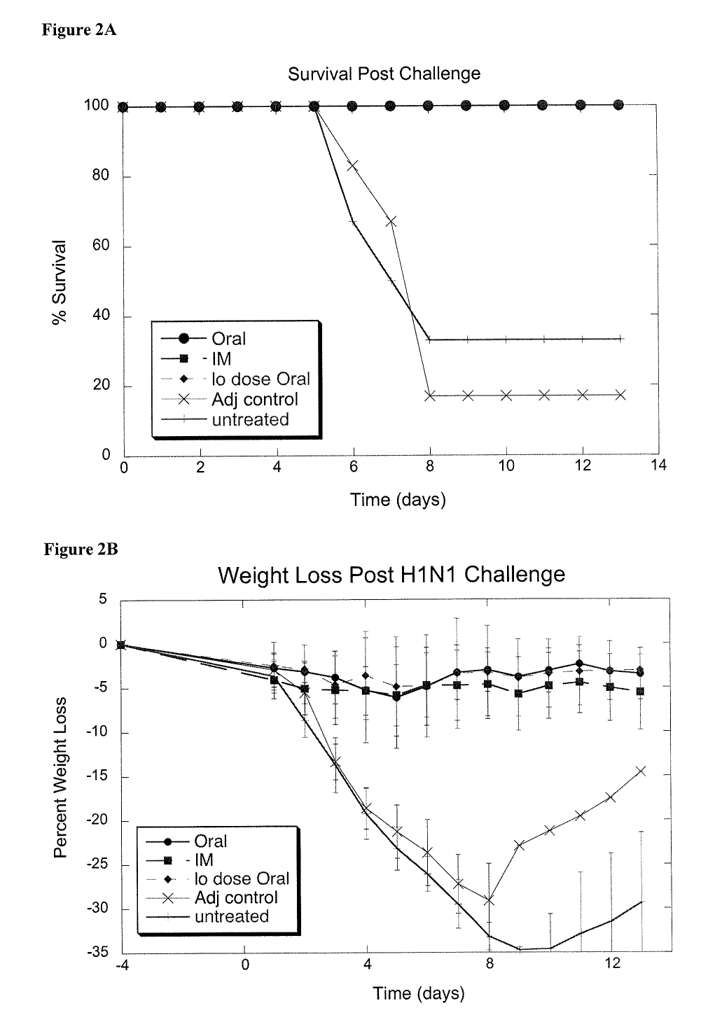

The results show that DX1-immunized animals were completely protected against death and substantial weight loss (FIGS. 2A and 2B). P<0.03 by Fisher's Exact Test when comparing DX1 groups to either untreated or adjuvant control groups for survival. The adjuvant control and untreated mice all lost significant amounts of weight and the majority died as a result of the challenge. Particularly significant are the data showing that a low oral dose of DX1 can still protect 100% of animals, with equivalent weight protection as i.m. immunized animals.

TABLE 1

| Group | Figure 2 Label | Description | N |
|---|---|---|---|
| A | Oral | 2× Oral immunization with 1e8 IU DX1 | 6 |
| B | IM | 2× IM immunization with 1e8 IU DX1 | 6 |
| C | lo dose Oral | 2× Oral immunization with 1e7 IU DX1 | 6 |
| D | Adj control | 2× Oral immunization 1e8 IU with an adjuvant control vector | 6 |
| E | untreated | untreated | 6 |

### Example 4

### Demonstration that Oral Delivery of DX1 is Superior to rAd5 for Eliciting Antibody Responses to H1N1 Influenza

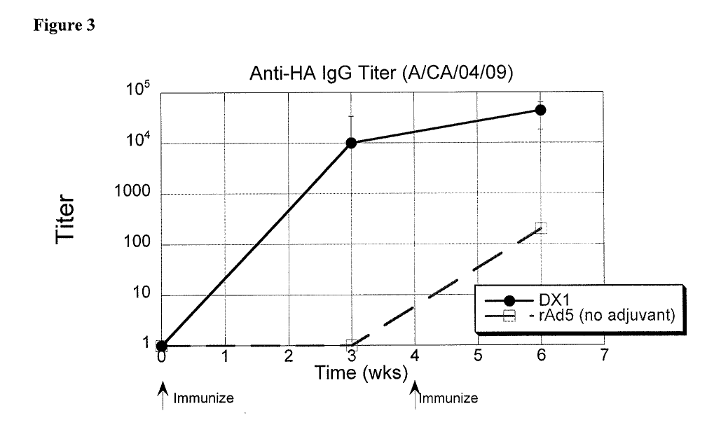

A second rAd vector was constructed that expresses the HA from A/CA/04/09, this time without the expressed dsRNA adjuvant. This vector was called DX2. Balb/c mice were dosed by oral gavage on weeks 0 and 4 with 1.0×10⁷ IU DX1 or DX2 (called rAd5 (no adjuvant) in FIG. 3). N=6 for this experiment. Antibody titers to A/CA/4/09 HA were measured in the plasma 3 and 7 weeks after administration by anti-HA IgG ELISA (FIG. 3). Results show that DX1 induced a superior antibody titer to DX2 demonstrating that the adjuvant improves immune responses to HA following oral delivery.

1. A chimeric adenoviral expression vector for the protection against H1N1 influenza, said vector comprising an expression cassette comprising:
(a) a first promoter operably linked to a nucleic acid encoding a toll-like receptor-3 (TLR-3) agonist, wherein the TLR-3 agonist is a double stranded RNA (dsRNA); and
(b) a second promoter operably linked to a nucleic acid encoding a polypeptide from H1N1 influenza.

2. The chimeric adenoviral expression vector of claim 1, wherein the nucleic acid encoding the TLR-3 agonist comprises a short hairpin RNA.

3. The chimeric adenoviral expression vector of claim 1, wherein the nucleic acid encoding the TLR-3 agonist comprises a sequence selected from the group consisting of SEQ ID NOs:1-6.

4. The chimeric adenoviral expression vector of claim 1, wherein the H1N1 influenza polypeptide is from A/CA/04/2009 H1N1 influenza.

5. The chimeric adenoviral expression vector of claim 1, wherein the H1N1 influenza polypeptide is selected from the group consisting of: HA, NA, MP, and NP.

6. The chimeric adenoviral expression vector of claim 5, wherein the H1N1 influenza polypeptide is an HA polypeptide.

7. The chimeric adenoviral expression vector of claim 1, wherein the first promoter and the second promoter are the same.

8. The chimeric adenoviral expression vector of claim 7, wherein the first promoter and the second promoter are each a CMV promoter.

9. The chimeric adenoviral expression vector according to claim 1, wherein said vector comprises a sequence having at least 95% identity to SEQ ID NO:7.

10. A pharmaceutical composition comprising the chimeric adenoviral expression vector of claim 1 and a pharmaceutically acceptable carrier, wherein said pharmaceutical composition is formulated for oral, mucosal, or intranasal administration.

11. An oral delivery pharmaceutical composition comprising the chimeric adenoviral expression vector according of claim 1 and a pharmaceutically acceptable carrier.

12. A method for eliciting an immune response to H1N1 influenza, said method comprising
administering to a mammal an immunogenically effective amount of the chimeric adenoviral expression vector of claim 1,
wherein the immune response is directed against the H1N1 influenza polypeptide, and wherein the route of administration is selected from the group consisting of: oral, intranasal, and mucosal.

13. The method of claim 12, wherein the route of administration is oral.

14. The method of claim 12, wherein the immune response is a mucosal immune response.

15. The method of claim 12, wherein said mammal is a human.
